(19) United States
(12) Patent Application Publication
(10) Pub. No.: US 2024/0050097 A1
(43) Pub. Date: Feb. 15, 2024

(54) ENDOVASCULAR COIL SPECIFICATION

(71) Applicant: KONINKLIJKE PHILIPS N.V., EINDHOVEN (NL)

(72) Inventors: Leili SALEHI, WALTHAM, MA (US); AYUSHI SINHA, BALTIMORE, MD (US); RAMON QUIDO ERKAMP, SWAMPSCOTT, MA (US); ASHISH PANSE, BURLINGTON, MA (US); GRZEGORZ ANDRZEJ TOPOREK, CAMBRIDGE, MA (US)

(21) Appl. No.: 18/266,674

(22) PCT Filed: Dec. 14, 2021
(86) PCT No.: PCT/EP2021/085698

§ 371 Date: Jun. 12, 2023

Related U.S. Application Data
(60) Provisional application No. 63/125,885, filed on Dec. 15, 2020.

(51) Int. Cl. A61B 17/12; G16H 30/20; G16H 30/40; G16H 50/20; G16H 50/30; A61B 34/10; A61B 90/00

(52) U.S. Cl. CPC A61B 17/12113; G16H 30/20; G16H 30/40; G16H 50/20; G16H 50/30; A61B 34/10; A61B 90/37; A61B 2034/108; A61B 2090/376

(57) ABSTRACT

A computer-implemented method of providing an endovascular coil specification of an endovascular coil for treating an aneurysm in a coil embolization procedure, includes: inputting (S120) X-ray image data (110), comprising one or more X-ray images including an aneurysm (120), into a neural network (130, 230) trained to predict, from the X-ray image data (110), endovascular coil data (140, 150) of an endovascular coil for treating the aneurysm (120); and outputting (S130) the endovascular coil data (140, 150) to provide the endovascular coil specification.

### TECHNICAL FIELD

The present disclosure relates to providing an endovascular coil specification for treating an aneurysm in a coil embolization procedure. A computer-implemented method, a processing arrangement, a system, and a computer program product, are disclosed.

### BACKGROUND

Aneurysms form at weak points in arterial walls and are evident in the form of a bulge or distension in the artery. Aneurysms that are at risk of rupture require treatment in order to avoid internal bleeding and/or haemorrhagic stroke. Endovascular coil embolization is a common procedure for treating intracranial cerebral aneurysms because it is performed in a minimally invasive manner and has a low failure rate. This procedure involves the insertion of deformable wires that form “coils” in the aneurysm in order to change the intra-aneurysmal hemodynamics; particularly the blood flow, and blood flow velocity into the aneurysm. The effect of the coils is to reduce shear stress on the aneurysm wall, and to promote thrombosis within the aneurysm, which can eventually seal off the aneurysm from the blood vessel.

The intra-aneurysmal hemodynamics depend on several anatomical factors such as the aneurysm type, for example whether it is bifurcated or not, the aneurysm position or angle relative to the blood flow, the curvature of parent vessel, the aneurysm neck diameter, and so forth. The intra-aneurysmal hemodynamics are also affected by procedural factors such as the coil packing density, and the residual aneurysm volume. A low coil packing density can result in recanalization and recurrence of the aneurysm, whereas a high coil packing can increase the risk of the aneurysm rupturing. A coil packing density of 20-25% is generally accepted as preventing recanalization in smaller aneurysms, although larger aneurysms may require a higher coil packing density in the range of 30-55% in order to reduce the risk of recanalization. The coil packing density is affected by aneurysm characteristics such as the aneurysm size, its neck diameter, and so forth, as well as physical properties of the coil such as the coil type, structure, material, coating, stiffness, diameter, and so forth.

Endovascular coil data is conventionally used in specifying a coil. Endovascular coils are typically defined by their type, namely: framing, filling, and finishing coils, and by their material. A coil embolization procedure starts with the insertion of one or more framing coils to fill the periphery of the aneurysmal sac, and thereby stabilize the structure for the subsequent insertion of filling coils. Filling coils are typically shorter and smaller, and are packed inside the framing coils. Lastly, finishing coils are inserted in order to finalize the treatment. Finishing coils are generally the softest and shortest of the coil types. Coils are primarily made from biocompatible inert materials such as nitinol, platinum, nickel, iridium, and tungsten, and are available with different coating materials. Typical coil coatings include bare platinum, polymer coated “Matrix”, and hydrophylic gel coated “HydroCoil”. The coating material may have an impact on aneurysm recurrence. As used herein, the term “characteristics” refers to the coil type, and its material.

Endovascular coils may also be defined by various “parameters”. The parameters define the coil length, the loop diameter, the coil stiffness. Coils used for endovascular embolization are available with different structures, including stock wire, primary wind, and secondary wind. Soft and small coils are known to be appropriate for the treatment of small aneurysms, whereas stiffer coils are generally used for large saccular aneurysms. Together, the endovascular coil characteristics, and the endovascular coil parameters, represent the endovascular coil data.

Specifying the correct endovascular coil, and moreover, the correct combination of endovascular coils for optimal embolization of different aneurysms, can however be challenging. A wide variety of coils are available. Most coils are designed to frame spherical aneurysms. Framing strategies for oblong aneurysms can differ significantly from those for spherical aneurysms. A mismatch between aneurysm geometry and framing strategy can result in increased aneurysm wall stress and introduce difficulties in creating a stable coil basket across the neck of the aneurysm. Knowledge of these differences, as well as the characteristics that are best suited for a specific aneurysm are key factors in ensuring effective embolization and successful procedural outcomes. However, building this knowledge requires several years of physician experience in different endovascular aneurysm treatment techniques.

Consequently, a need exists for an improved method of specifying an endovascular coil for treating an aneurysm in a coil embolization procedure.

### SUMMARY

According to a first aspect of the present disclosure a computer-implemented method of providing an endovascular coil specification of an endovascular coil for treating an aneurysm in a coil embolization procedure, is provided. The method includes:
receiving X-ray image data, the X-ray image data comprising one or more X-ray images including an aneurysm;
inputting the X-ray image data, into a neural network trained to predict, from the X-ray image data, endovascular coil data of an endovascular coil for treating the aneurysm; and
outputting the endovascular coil data to provide the endovascular coil specification.

According to a second aspect of the present disclosure, a computer-implemented method of training a neural network for providing an endovascular coil specification of an endovascular coil for treating an aneurysm in a coil embolization procedure, is provided. The method includes:
receiving X-ray image training data, the X-ray image training data comprising one or more X-ray images including an aneurysm;
receiving ground truth endovascular coil specification data representing endovascular coil data of an endovascular coil used to treat the aneurysm in the X-ray image training data;
inputting the received X-ray image training data, into the neural network, and adjusting parameters of the neural network based on a loss function representing a difference between the endovascular coil data, predicted by the neural network, and the endovascular coil data of the endovascular coil used to treat the aneurysm in the X-ray image training data represented by the received ground truth endovascular coil specification data.

Further aspects, features and advantages of the present disclosure will become apparent from the following description of examples, which is made with reference to the accompanying drawings.

### BRIEF DESCRIPTION OF THE DRAWINGS

FIG. 1 illustrates an example X-ray image including an aneurysm 120.

FIG. 2 is a flowchart of an example method of providing an endovascular coil specification for treating an aneurysm in a coil embolization procedure, in accordance with some aspects of the disclosure.

FIG. 3 is a schematic diagram illustrating a first example neural network 130 for providing an endovascular coil specification for treating an aneurysm in a coil embolization procedure, in accordance with some aspects of the disclosure.

FIG. 4 is a schematic diagram illustrating a second example neural network 130 for providing an endovascular coil specification for treating an aneurysm in a coil embolization procedure, in accordance with some aspects of the disclosure.

FIG. 5 is a schematic diagram illustrating a third example neural network 230 for providing an endovascular coil specification for treating an aneurysm in a coil embolization procedure, in accordance with some aspects of the disclosure.

FIG. 6 is a flowchart of an example method of training a neural network for providing an endovascular coil specification for treating an aneurysm in a coil embolization procedure, in accordance with some aspects of the disclosure.

FIG. 7 is a schematic diagram illustrating an example method of training a neural network 130 for providing an endovascular coil specification for treating an aneurysm in a coil embolization procedure, in accordance with some aspects of the disclosure.

FIG. 8 is a schematic diagram illustrating an example system 300 for providing an endovascular coil specification for treating an aneurysm in a coil embolization procedure, in accordance with some aspects of the disclosure.

### DETAILED DESCRIPTION

Examples of the present disclosure are provided with reference to the following description and the figures. In this description, for the purposes of explanation, numerous specific details of certain examples are set forth. Reference in the specification to “an example”, “an implementation” or similar language means that a feature, structure, or characteristic described in connection with the example is included in at least that one example. It is also to be appreciated that features described in relation to one example may also be used in another example, and that all features are not necessarily duplicated in each example for the sake of brevity. For instance, features described in relation to a computer-implemented method may be implemented in a processing arrangement, and in a system, and in a computer program product, in a corresponding manner.

In the following description, reference is made to computer implemented methods that involve a coil embolization procedure. Reference is made to coil embolization procedures within the brain. However, it is to be appreciated that the method of providing an endovascular coil specification as described herein may find application in specifying an endovascular coil for use in treating aneurysms in coil embolization procedures in other regions of the vasculature, such as for example the thoracic aorta, the abdominal aorta, in the neck, ovaries, arm or leg, and so forth.

It is noted that the computer-implemented methods disclosed herein may be provided as a non-transitory computer-readable storage medium including computer-readable instructions stored thereon which, when executed by at least one processor, cause the at least one processor to perform the method. In other words, the computer-implemented methods may be implemented in a computer program product. The computer program product can be provided by dedicated hardware or hardware capable of running the software in association with appropriate software. When provided by a processor, or “processing arrangement”, the functions of the method features can be provided by a single dedicated processor, by a single shared processor, or by a plurality of individual processors, some of which can be shared. The explicit use of the terms “processor” or “controller” should not be interpreted as exclusively referring to hardware capable of running software, and can implicitly include, but is not limited to, digital signal processor “DSP” hardware, read only memory “ROM” for storing software, random access memory “RAM”, a non-volatile storage device, and the like. Furthermore, examples of the present disclosure can take the form of a computer program product accessible from a computer usable storage medium or a computer-readable storage medium, the computer program product providing program code for use by or in connection with a computer or any instruction execution system. For the purposes of this description, a computer-usable storage medium or computer-readable storage medium can be any apparatus that can comprise, store, communicate, propagate, or transport a program for use by or in connection with an instruction execution system, apparatus, or device. The medium can be an electronic, magnetic, optical, electromagnetic, infrared, or semiconductor system or device or propagation medium. Examples of computer-readable media include semiconductor or solid-state memories, magnetic tape, removable computer disks, random access memory “RAM”, read only memory “ROM”, rigid magnetic disks, and optical disks. Current examples of optical disks include compact disk-read only memory “CD-ROM”, optical disk-read/write “CD-R/W”, Blu-Ray™, and DVD.

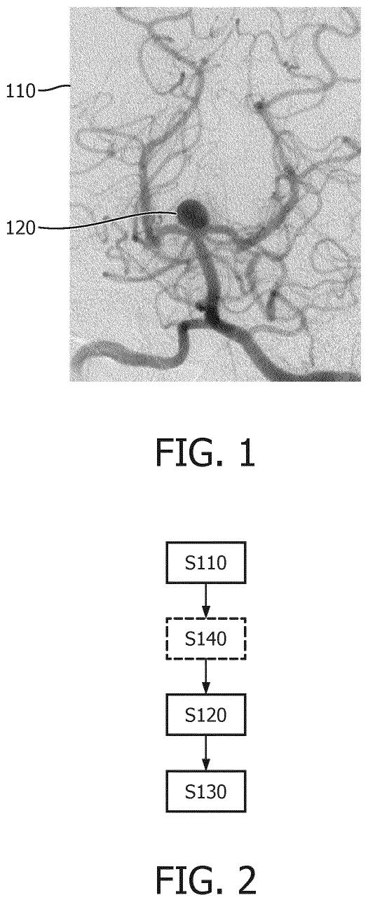

FIG. 1 illustrates an example X-ray image including an aneurysm 120. The X-ray image in FIG. 1 is a digital subtraction angiography “DSA” image of the brain, and indicates the vasculature by virtue of the use of a radiopaque contrast agent. The brain aneurysm 120 illustrated in FIG. 1 is at the top of the basilar artery, and without treatment, may rupture and result in a subarachnoid haemorrhage at the base of the brain. The aneurysm 120 in FIG. 1 may be treated using endovascular coil embolization wherein wires are inserted in the aneurysm 120 to form “coils” in the aneurysm, and thereby change the intra-aneurysmal hemodynamics.

The inventors have determined a method of providing an endovascular coil specification of an endovascular coil for treating an aneurysm in a coil embolization procedure. The method may be used to provide a specification of an endovascular coil for treating the example brain aneurysm 120 illustrated in FIG. 1, as well as other aneurysms. The method is described with reference to FIG. 2, which is a flowchart of an example method of providing an endovascular coil specification for treating an aneurysm in a coil embolization procedure, in accordance with some aspects of the disclosure. The method may be implemented by a computer, and includes:
receiving S110 X-ray image data 110, the X-ray image data 110 comprising one or more X-ray images including an aneurysm 120;
inputting S120 the X-ray image data 110, into a neural network 130, 230 trained to predict, from the X-ray image data 110, endovascular coil data 140, 150 of an endovascular coil for treating the aneurysm 120; and
outputting S130 the endovascular coil data 140, 150 to provide the endovascular coil specification.

The X-ray image(s) included in the X-ray image data 110 may for example include an X-ray image such as the X-ray image illustrated in FIG. 1. In general, the X-ray image data 110 may include one or more X-ray fluoroscopy images, and/or one or more contrast-enhanced X-ray image(s), such as DSA images.

The X-ray image data 110 received in the FIG. 2 method may be received from various sources, including a database, an X-ray imaging system, a computer readable storage medium, the cloud, and so forth. The data may be received using any form of data communication, such as wired or wireless data communication, and may be via the internet, an ethernet, or by transferring the data by means of a portable computer-readable storage medium such as a USB memory device, an optical or magnetic disk, and so forth.

In operation S120, the X-ray image data 110 is inputted into the neural network 130 that is trained to predict, from the X-ray image data 110, endovascular coil data 140, 150 of an endovascular coil for treating the aneurysm 120. In some examples, the neural network in inputted with un-segmented X-ray image date 110. In these examples, the neural network identifies features of the aneurysm relevant to its predictions, from the un-segmented X-ray image date 110. In other examples, the neural network in inputted with segmented X-ray image date 110. In these examples, the aneurysm, and optionally the vasculature, are identified by segmenting the X-ray image data 110 in a segmentation operation S140 prior to inputting the X-ray image data into the neural network 130. The segmentation operation S140 is therefore optional, as indicated in FIG. 1 by way of the dashed outline to item S140.

The segmentation performed in operation S140 may include the use of a bounding box, a mesh, a centroid, a binary segmentation, and so forth. The segmentation may be performed using one or more known techniques such as: thresholding, region growing, template matching, level sets, active contour modelling, neural networks (e.g., U-Nets), manual or semi-automatic annotation/segmentation/detection methods, and so forth.

When the segmentation operation S140 is performed, the method of providing the endovascular coil specification includes: segmenting S140 the X-ray image data 110 to identify the aneurysm 120, prior to inputting S120 the X-ray image data 110, into the neural network 130; and the inputting S120 the X-ray image data 110, into a neural network 130, comprises inputting the segmented X-ray image data 110 into the neural network.

In some examples, the X-ray image data 110 inputted into the neural network 130 in operation S120 may include one and only one X-ray image. In other examples, the X-ray image data 110 inputted into the neural network 130 in operation S120 includes multiple X-ray images. When multiple X-ray images are inputted, the X-ray images may include i) multiple different viewing angles of the aneurysm 120 and/or ii) they may represent different timesteps during the coil embolization procedure. As compared to using a single X-ray image, the accuracy of the predictions made by the neural network 130 may be improved by inputting X-ray images having different viewing angles. Additional viewing angles may assist the neural network 130 to assess aneurysm features such as its volume and neck diameter. The use of X-ray images with near-orthogonal oriented views may be considered to provide higher accuracy than similarly-oriented views.

The accuracy of the predictions made by the neural network may also be improved by inputting X-ray images that represent different timesteps during the coil embolization procedure. Intra-procedural X-ray images include valuable information about the progress of the procedure and provide information for the neural network to base its predictions on. By inputting a combination of pre-procedural, and intra-procedural X-ray images into the neural network, the neural network may learn to associate the progress of the procedure, as well as the current state of the embolism with the endovascular coil data of the endovascular coil that should be used in a next step of the procedure.

The endovascular coil data that is predicted by the neural network 130 in operation S120, may include one or more endovascular coil parameters 140 for treating the aneurysm 120, and/or one or more characteristics 150 of an endovascular coil for treating the aneurysm 120. The one or more endovascular coil parameters 140 may include one or more of: a coil length, a coil diameter, a coil stiffness, and a coil loop diameter. The one or more characteristics 150 of the endovascular coil may include one or more of: a coil type, and a coil material. Other endovascular coil parameters, and characteristics may also be predicted by the neural network 130.

In operation S130, the endovascular coil data 140, 150 is outputted to provide the endovascular coil specification. The outputting in operation S130 may include displaying the endovascular coil data on a display, storing the endovascular coil data to a computer-readable storage device, and so forth. In so doing, an endovascular coil specification is provided that is suited to the aneurysm in the X-ray image data.

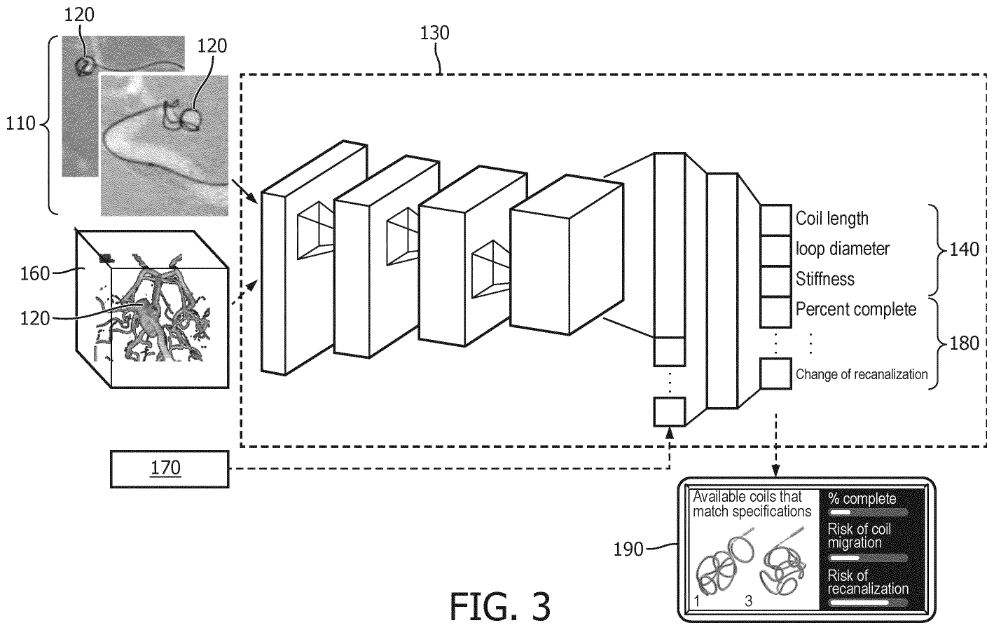

The use of various types of neural networks in the neural network, is contemplated. FIG. 3 is a schematic diagram illustrating a first example neural network 130 for providing an endovascular coil specification for treating an aneurysm in a coil embolization procedure, in accordance with some aspects of the disclosure. The example neural network 130 illustrated in FIG. 3 is suited to predicting parameters 140 such as a coil length, a coil diameter, a coil stiffness, and a coil loop diameter. The example neural network 130 illustrated in FIG. 3 includes multiple fully connected or convolutional layers with pooling, normalization, non-linear layers, and so forth between them. Non-linear layers or activation functions for this type of network include, but are not limited to, ReLU, leaky ReLU, linear activation, sigmoid, tanh, etc. The example neural network 130 generates an n×1 output vector, wherein n is the number of predictions. The illustrated example predictions include endovascular coil parameters 140 such as coil length, loop diameter and stiffness. As described in more detail below, the predictions made by the neural network 130 may optionally additionally include procedural outcome data 180 consequent to using the predicted endovascular coil data to treat the aneurysm 120 in the coil embolization procedure.

During inference, the example neural network 130 in FIG. 3 is inputted with X-ray image data 110, which in the illustrated example includes multiple intra-procedural X-ray images that include an aneurysm 120. Predictions are then made by the neural network from the inputted X-ray image data 110. Whilst FIG. 3 illustrates predictions being made from multiple intra-procedural X-ray images, it is noted that in some examples, a single X-ray image may alternatively be inputted into the neural network 130 in order to make its predictions.

In the example illustrated in FIG. 3, the predictions made by the neural network 130 include predictions of the endovascular coil parameters 140. The endovascular coil parameters 140 are then outputted in order to provide an endovascular coil specification. The endovascular coil parameters 140 may for example be outputted to the illustrated display 190, or to a computer-readable storage device.

In some examples, the method of providing an endovascular coil specification also includes identifying one or more endovascular coils for treating the aneurysm in the coil embolization procedure. This is illustrated on the left side of the display 190 in the lower portion of FIG. 3. This operation may be performed within, or outside the neural network 130. In this operation, the method of providing an endovascular coil specification includes:
comparing the outputted endovascular coil data 140 with a database of endovascular coil data for each of a plurality of endovascular coils; and
identifying one or more of the plurality of endovascular coils for treating the aneurysm in the coil embolization procedure, based on the comparing.

The comparing may for example include using a lookup table to identify from an inventory of coils, one or more coils that satisfy the coil length and stiffness predicted by the neural network 130. The identifying in this operation may for example include displaying icons of the identified one or more coils on a display, displaying model numbers of the identified one or more coils, and so forth.

FIG. 3 also illustrates by way of dashed lines that volumetric image data 160 may optionally also be inputted to the neural network 130. The volumetric image data 160 represents the aneurysm 120 in the X-ray image data 110. The volumetric image data 160 may be provided by various imaging modalities, and may for example represent one or more CT images, one or more 3D rotational angiograms “3DRA”, one or more computed tomography angiograms “CTA”, one or more magnetic resonance angiograms “MRA”. The volumetric image data 160 may alternatively be provided by an intravascular ultrasound “IVUS” imaging system and represent IVUS imaging data. The volumetric image data 160 may be generated prior to a coil embolization procedure, or during a coil embolization procedure. In other words, the volumetric image data may be pre-procedural or intra-procedural volumetric image data. In this example, during inference, the method of providing an endovascular coil specification, includes:

receiving volumetric image data 160 representing the aneurysm 120 in the X-ray image data 110;

inputting the received volumetric image data 160 into the neural network 130;

and predicting the endovascular coil data 140, from the received X-ray image data 110, and from the received volumetric image data 160.

FIG. 3 also illustrates by way of dashed lines that patient data 170 may optionally also be inputted into the neural network 130. The patient data 170 corresponds to the aneurysm 120 in the X-ray image data 110. The patient data may for example include one or more of: the patient age, gender, weight, smoking history, genomic data, type of the aneurysm 120, measurements of the aneurysm such as volume and neck diameter, and so forth. During inference, the neural network 130 may thus make its predictions from the inputted patient data 170, as well as the inputted X-ray image data 110. The patient data 170 may be incorporated into the neural network 130 by appending it to the flattened feature layers, before applying linear transformations. In this example, during inference, the method of providing an endovascular coil specification, includes:

receiving patient data 170 corresponding to the aneurysm 120;

inputting the received patient data 170 into the neural network 130; and

predicting the endovascular coil data 140, from the received X-ray image data 110, and from the received patient data 170.

As mentioned above, and as illustrated in FIG. 3, the predictions made by the neural network 130 may additionally include procedural outcome data 180 consequent to using the predicted endovascular coil data to treat the aneurysm 120 in the coil embolization procedure. The predicted procedural outcome data 180 may include factors such as the percentage completion of filling the aneurysm with coils “Percent complete”, and the “Chance of recanalization”. The predicted procedural outcome data 180 may alternatively or additionally include factors such as a predicted risk of rupture and/or a recommended follow-up interval. As described in more detail below, the neural network is predicted to generate the procedural outcome data 180 by training the neural network with inputted ground truth procedural outcome data.

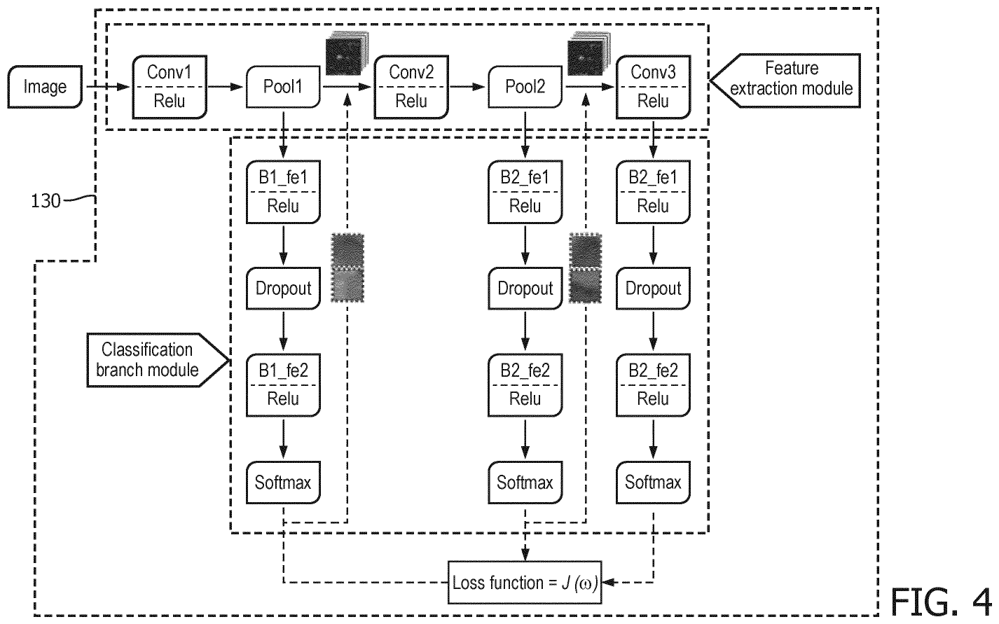

FIG. 4 is a schematic diagram illustrating a second example neural network 130 for providing an endovascular coil specification for treating an aneurysm in a coil embolization procedure, in accordance with some aspects of the disclosure. The example neural network 130 illustrated in FIG. 4 is suited to predicting parameters 140 such as a coil length, a coil diameter, a coil stiffness, and a coil loop diameter. In comparison to classification or segmentation tasks, regression tasks such as the prediction of parameters 140 such as a coil length, a coil diameter, a coil stiffness, and a coil loop diameter, are typically more difficult, especially for complex outcome distributions. In the example neural network illustrated in FIG. 4, classification and regression tasks are combined. This narrows the scope within which the neural network must regress the many variables. The neural network 130 illustrated in FIG. 4 includes a cascaded convolutional neural network wherein a weighted loss function J(ω) is used. The weighted loss function J(ω) defines a focus of the network on the different coarse and/or fine outputs. In the illustrated example, a coarse feature can be used for a less complex classification task such as differentiating coil stiffness by binning coil stiffness into different classes of stiffness, and a fine feature with a more complex classification task is the regression task of determining parameters within these bins. By reducing the space of parameter regression, the efficiency of training the network can be improved. During training, the parameters of the FIG. 4 neural network may be adjusted by firstly training a coarse branch of the neural network on a classification task, and after the generalizability of the neural network is obtained, the weights of the coarse branch are frozen. Secondly, a fine branch which derives from the coarse branch, is trained on the regression task within the class with the highest response in the coarse branch. It is assumed that features learned on the less complex task can be reused for the more complex regression task.

Another optional input into the neural network 130 may be the characteristics of the endovascular coil. For example, the neural network may be provided with input that the next coil to be inserted into the aneurism will be e.g. a filling coil. This can provide additional context to the neural network 130 in its prediction of parameters such as coil length, etc. In this example, during inference, the method of providing an endovascular coil specification, includes:
receiving characteristics of an endovascular coil to be inserted into the aneurysm 120;
inputting the received characteristics of the endovascular coil into the neural network 130; and
predicting the endovascular coil data 140, from the received X-ray image data 110, and from the received characteristics of the endovascular coil.

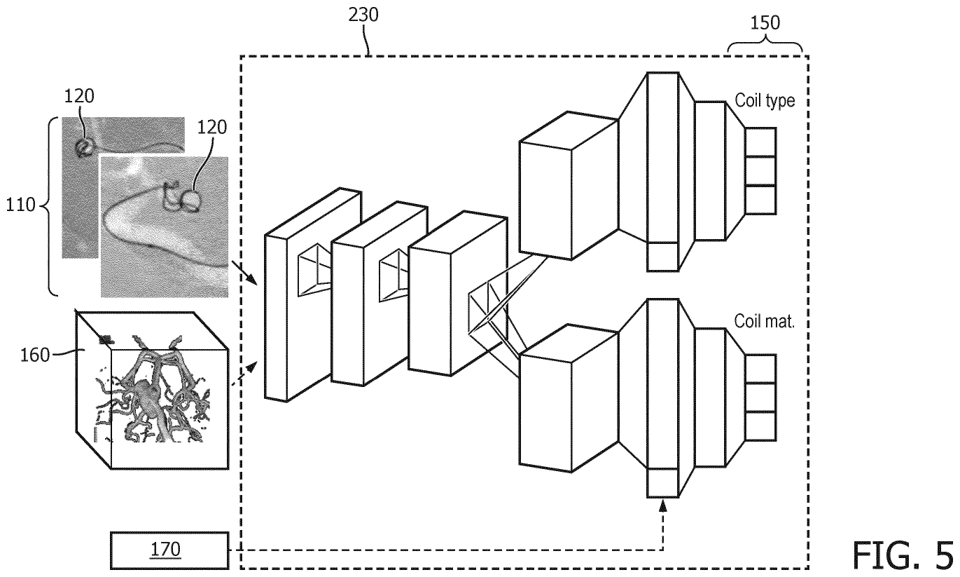

In some examples, the characteristics of the endovascular coil may be received as user input. For example, it may be obtained by a user manually selecting the characteristics of the next coil to be inserted into the aneurysm via a dropdown menu, or via touchscreen selection, and so forth. In other examples, the characteristics of the endovascular coil may be generated automatically. For example, a camera in the operating room may observe the endovascular coils being prepared for the next step in the procedure and automatically detect their characteristics. In another example, the current status of the procedure may be used to predict the characteristics of the next endovascular coil to be inserted into the aneurysm. This example is illustrated in FIG. 5.

FIG. 5 is a schematic diagram illustrating a third example neural network 230 for providing an endovascular coil specification for treating an aneurysm in a coil embolization procedure, in accordance with some aspects of the disclosure. The example neural network 230 illustrated in FIG. 5 is suited to predicting characteristics 150 of an endovascular coil for treating the aneurysm 120 such as a coil type, and a coil material. Items in FIG. 5 having the same reference number as FIG. 3 perform functions corresponding to those described above with reference to FIG. 3. Thus, during inference, the neural network 230 is inputted with X-ray image data 110 and is trained to predict from the X-ray image data 110, endovascular coil data 150 of an endovascular coil for treating the aneurysm 120. The neural network 230 outputs the endovascular coil data 150 to provide an endovascular coil specification. As described in relation to FIG. 3, the neural network 230 may additionally be inputted with volumetric image data 160, and predict the endovascular coil data 150, from the received X-ray image data 110, and from the received volumetric image data 160. Likewise, the neural network 230 may additionally be inputted with patient data 170, and predict the endovascular coil data 150, from the received X-ray image data 110, and from the received patient data 170.

The neural network 230 illustrated in FIG. 5 represents a multi-label classification network, otherwise known as a multi-output-multi-class, or a multi-task, classification network, and is trained to predict characteristic such as the coil type and material to use in the next step of the coil embolization procedure. The illustrated neural network 230 includes multiple fully connected and/or convolutional layers with pooling, drop-out, normalization, non-linear layers, etc. between them. The neural network 230 in FIG. 5 includes multiple shared convolutional layers that are shared between the classification outputs “coil type” and “coil material”. The output of the final shared layer is then inputted into a separate input layer for each classification task. A Softmax activation function may be used in the output layer. In the illustrated example, the classification outputs are “coil type” and “coil material”. Other classifications may be predicted in a similar manner. As mentioned above in relation to FIG. 3, the neural network illustrated in FIG. 5 may also be used to identify one or more endovascular coils for treating the aneurysm in the coil embolization procedure by comparing the characteristics 150 of the endovascular coil outputted by the neural network 230, with a database of endovascular coil characteristics for each of a plurality of endovascular coils.

As illustrated in FIG. 5, the optional patient data 170 may be incorporated into the neural network 230 by appending it to the flattened feature layers for each classification before applying a linear transformation and then non-linearities (e.g., Softmax, sigmoid, logistic activation, tanh, etc.) to obtain the classification of “coil type” and “coil material”. Alternatively, the patient data 170 may be repeated or tiled and concatenated, added, or multiplied, etc. with any of the intermediate feature maps. In an alternative to the FIG. 5 neural network architecture, the neural network 230 may have a single branch wherein the classification outputs “coil type” and “coil material” are combined into a single multi-label classification task. In this case, a multi-label classification network is trained to predict two labels, i.e. coil material and coil type, using a sigmoid, rather than a Softmax, activation function.

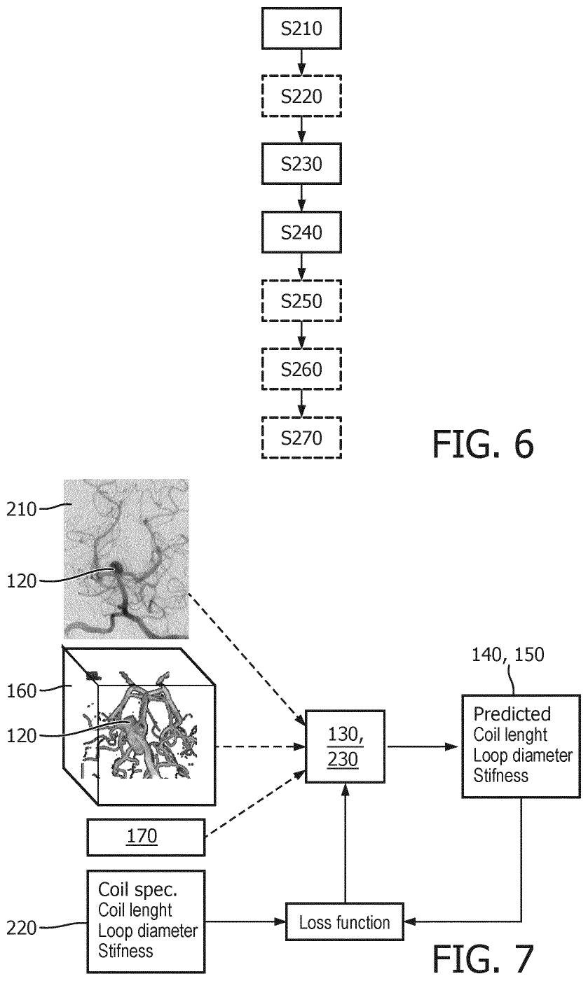

The neural networks described above with reference to FIG. 3-FIG. 5 may be trained by inputting training data into the neural network and adjusting the parameters, or more specifically the weights and biases of the neural network, based on a loss function. The loss function computes an error value representing a difference between the predicted output of the neural network, and a ground truth, or expected output, of the neural network. The parameters are adjusted iteratively by inputting the training data, computing the value of the loss function, and adjusting the parameters until a stopping criterion is met. The stopping criterion may represent that the value of the loss function is within a predetermined range. The training of the neural networks described above with reference to FIG. 3-FIG. 5 is described with reference to FIG. 6 and FIG. 7. FIG. 6 is a flowchart of an example method of training a neural network for providing an endovascular coil specification for treating an aneurysm in a coil embolization procedure, in accordance with some aspects of the disclosure. With reference to FIG. 6, a method of training the above-described neural networks 130, 230 includes training the neural network 130, 230 to predict the endovascular coil data 140, 150 of the endovascular coil, by:

receiving S210 X-ray image training data 210, the X-ray image training data 210 comprising one or more X-ray images including an aneurysm 120;

receiving S220 ground truth endovascular coil specification data 220 representing endovascular coil data of an endovascular coil used to treat the aneurysm 120 in the X-ray image training data 210;

inputting S230 the received X-ray image training data 210, into the neural network 130, 230, and adjusting S240 parameters of the neural network 130, 230 based on a loss function representing a difference between the endovascular coil data 140, 150, predicted by the neural network 130, 230, and the endovascular coil data of the endovascular coil used to treat the aneurysm 120 in the X-ray image training data 210 represented by the received ground truth endovascular coil specification data 220.

Thus, in the above method, the expected output of the neural network 130, 230, is the ground truth endovascular coil specification data 220 representing endovascular coil data of an endovascular coil used to treat the aneurysm 120 in the X-ray image training data 210. For example, if the predicted endovascular coil data 140, 150 includes a parameter such as the coil length, the coil length in the ground truth endovascular coil specification data 220 is used to compute the value of the loss function, and thereby train the neural network 130. By way of another example, if the predicted endovascular coil data 140, 150 includes a characteristic such as the coil type, the coil type in the ground truth endovascular coil specification data 220 is used to compute the value of the loss function, and thereby train the neural network 230.

FIG. 6 also illustrates that in addition to the above-described operations S210, S220, S230 and S240; that operations S250, S260 and S270 may optionally additionally be performed. These latter operations are described in more detail below.

FIG. 7 is a schematic diagram illustrating an example method of training a neural network 130 for providing an endovascular coil specification for treating an aneurysm in a coil embolization procedure, in accordance with some aspects of the disclosure. FIG. 7 illustrates schematically the inputting of X-ray image training data 210, into the neural network 130, 230. The X-ray image training data 210 represents X-ray image data 110 similar to that which is inputted to the neural network 130, 230 during inference, and additionally includes corresponding ground truth endovascular coil specification data 220 such as coil length, loop diameter, and stiffness. The loss function in FIG. 7 computes a difference between the predicted endovascular coil data 140, 150, and the ground truth endovascular coil specification data 220, and the value of this difference, or error, is used to adjust the parameters of the neural network 130, 230. Loss functions such as the negative log-likelihood loss, binary cross entropy loss, categorical cross-entropy, Hinge loss, the L1 or L2 loss, Huber loss, log cosh loss, and so forth, may be used.

During training, the value of the loss function is typically minimized, and training is terminated when the value of the loss function satisfies a stopping criterion. Sometimes, training is terminated when the value of the loss function satisfies one or more of multiple criteria. Various methods are known for solving this minimization problem such as gradient descent, Quasi-Newton methods, and so forth. Various algorithms have been developed to implement these methods and their variants including but not limited to Stochastic Gradient Descent “SGD”, batch gradient descent, mini-batch gradient descent, Gauss-Newton, Levenberg Marquardt, Momentum, Adam, Nadam, Adagrad, Adadelta, RMSProp, and Adamax “optimizers”.

These algorithms compute the derivative of the loss function with respect to the model parameters using the chain rule. This process is called backpropagation since derivatives are computed starting at the last layer or output layer, moving toward the first layer or input layer. These derivatives inform the algorithm how the model parameters must be adjusted in order to minimize the error function. That is, adjustments to model parameters are made starting from the output layer and working backwards in the network until the input layer is reached. In a first training iteration, the initial weights and biases are often randomized. The neural network then predicts the output data, which is likewise, random. Backpropagation is then used to adjust the weights and the biases. The training process is performed iteratively by making adjustments to the weights and biases in each iteration. Training is terminated when the error, or difference between the predicted output data and the expected output data, is within an acceptable range for the training data, or for some validation data. Subsequently the neural network may be deployed, and the trained neural network makes predictions on new input data using the trained values of its parameters. If the training process was successful, the trained neural network accurately predicts the expected output data from the new input data.

As mentioned above, during inference, in some examples the neural network 130, 230, may optionally predict the endovascular coil data 140, 150 from volumetric image data 160, as well as from X-ray image data. In these examples, during training, the neural network is inputted with volumetric image training data 260. This is indicated in FIG. 7 by way of the dashed lines connecting volumetric image training data 260 with the neural network 130, 230. The volumetric image training data 260 is the same type of data as the volumetric image data 260 that is inputted into the trained neural network during inference. The volumetric image training data 260 represents the aneurysm 120 in the X-ray image training data 210. In other words, the volumetric image training data 260 includes the same aneurysm that is in the X-ray image(s) in the X-ray image training data 210. The volumetric image training data 260 originates from the same source as the source of the volumetric image data 160 that is inputted during inference, and may therefore likewise be provided by various imaging systems. The volumetric image training data 260 may represent one or more CT images, one or more 3D rotational angiograms “3DRA”, one or more computed tomography angiograms “CTA”, one or more magnetic resonance angiograms “MRA”, or represent IVUS imaging data, and so forth.

In these examples, the neural network 130, 230 is trained to predict the endovascular coil data 140, 150 of the endovascular coil for treating the aneurysm 120, from the X-ray image data 110, and from volumetric image data 160 representing the aneurysm 120 in the X-ray image data 110; and the neural network 130, 230 is trained to predict the endovascular coil data 140, 150 of the endovascular coil, by further:
receiving S250 volumetric image training data 260 representing the aneurysm 120 in the X-ray image training data 210;
inputting S260 the received volumetric image training data 260 into the neural network 130, 230; and
predicting S270 the endovascular coil data 140, 150 of the endovascular coil for treating the aneurysm 120, from the received X-ray image training data 210, and from the received volumetric image training data 260.

As mentioned above, during inference, in some examples the neural network 130, 230, may optionally predict the endovascular coil data 140, 150 from patient data 170, as well as from X-ray image data. In these examples, during training, the neural network is inputted with patient training data 270. This is indicated in FIG. 7 by way of the dashed lines connecting patient training data 270 with the neural network 130, 230. The patient training data 270 is the same type of data as the patient data that is inputted into the trained neural network during inference. The patient data may therefore include one or more of: the patient age, gender, weight, smoking history, genomic data, type of the aneurysm 120, measurements of the aneurysm such as volume and neck diameter, and so forth. Moreover, the patient training data 270 corresponds to the aneurysm 120 in the X-ray image training data 210. In other words, the patient training data 270 originates from the same patient as the X-ray image(s) in the X-ray image training data 210.

In these examples, the neural network 130, 230 is further trained to predict the endovascular coil data 140, 150 of the endovascular coil for treating the aneurysm 120, from patient data 170 corresponding to the aneurysm 120 in the X-ray image data 110; and the neural network 130, 230 is trained to predict the endovascular coil data 140, 150 of the endovascular coil for treating the aneurysm 120, by further:
receiving patient training data 270 corresponding to the aneurysm 120 in the X-ray image training data 210;
inputting the received patient training data 270 into the neural network 130, 230; and
predicting the endovascular coil data 140, 150 of the endovascular coil for treating the aneurysm 120, based further on the received patient training data 270.

As mentioned above, during inference, in some examples, the predictions made by the neural network 130 may additionally include procedural outcome data 180 consequent to using the predicted endovascular coil data to treat the aneurysm 120 in the coil embolization procedure. In these examples, during training, ground truth procedural outcome data is inputted into the loss function and used to adjust parameters of the neural network. The ground truth procedural outcome data is the same type of data as the procedural outcome data 180 that is predicted by the trained neural network during inference. The ground truth procedural outcome data may therefore include factors such as the percentage completion of filling the aneurysm with coils “Percent complete”, and the “Chance of recanalization”. The ground truth procedural outcome data represents an outcome of using the ground truth endovascular coil specification data 220 to treat the aneurysm 120 in the X-ray image training data 210.

In these examples, the neural network 130, 230 may be further trained to predict the endovascular coil data 140, 150 of the endovascular coil for treating the aneurysm 120, by further;
receiving ground truth procedural outcome data representing an outcome of using the ground truth endovascular coil specification data 220 to treat the aneurysm 120 in the X-ray image training data 210;
wherein the adjusting S240 parameters of the neural network 130, 230 comprises reducing a value of the loss function; and
wherein a negative procedural outcome is configured to increase a value of the loss function.

By penalizing the loss function in this manner, it is provided that negative outcomes are used to avoid the neural network making predictions that may have an adverse patient outcome.

In examples wherein the predictions made by the neural network 130 include procedural outcome data 180, the neural network 130, 230 may be further trained to predict, from the X-ray image data 110, procedural outcome data 180 representing at least one of:

a fractional value representing the completeness of the coil embolization procedure;
a risk of rupture of the aneurysm 120;
a risk of recanalization of the aneurysm 120;
a recommended follow-up interval;
consequent to using the predicted endovascular coil data 140, 150 of the endovascular coil to treat the aneurysm 120 in the coil embolization procedure.

In these examples the neural network 130, 230 is trained to predict the procedural outcome data 180 by:
receiving ground truth procedural outcome data representing an outcome of using the ground truth endovascular coil specification data 220 to treat the aneurysm 120 in the X-ray image training data 210; and inputting the received ground truth procedural outcome data, into the neural network 130, 230;
and wherein the loss function used in the adjusting S240 parameters of the neural network 130, 230 is based further on a difference between the procedural outcome data 180 predicted by the neural network 130, 230, and the received ground truth procedural outcome data.

Any of the above-described methods may additionally include the computing, and optional outputting a confidence estimate of the endovascular coil data 140, 150, predicted by the neural network 130, 230. A confidence estimate may be based on various factors. In one example a confidence estimate may be determined based on a location of the attention, i.e. an “attention map” indicating the regions of the X-ray image data 110 on which the predictions of the neural network 130, 230, were made. For instance, if the attention of the network was within the aneurysm 120, a relatively higher level of confidence may be provided, whereas if the attention of the network was further from the aneurysm 120, a relatively lower level of confidence may be provided. Another indicator of confidence may be the number of X-ray images in the X-ray image data 110 that are used to produce the predictions. For instance, at the start of a coil embolization procedure, and when there are no coils in the aneurysm, a single X-ray image might produce a confidence estimate. As the procedure continues, however, the distribution of coil and the residual space within the aneurysm becomes harder to estimate from a single image. The confidence estimate of the network, may therefore, decrease if only a single X-ray image is used by the neural network to generate the predictions at a later stage in the coil embolization procedure. The confidence estimate during the coil embolization procedure may increase if additional X-ray images are used by the neural network to generate the predictions. The confidence estimate may also be determined based on whether the additional information such as the coil type or coil material, are used as inputs to the neural network. Similarly, if patient data such as the patient's smoking history is available, this increases the confidence estimate of the neural network's prediction of risk of rupture due to the strong association between this risk and smoking history, which can thin the walls of the aneurysm.

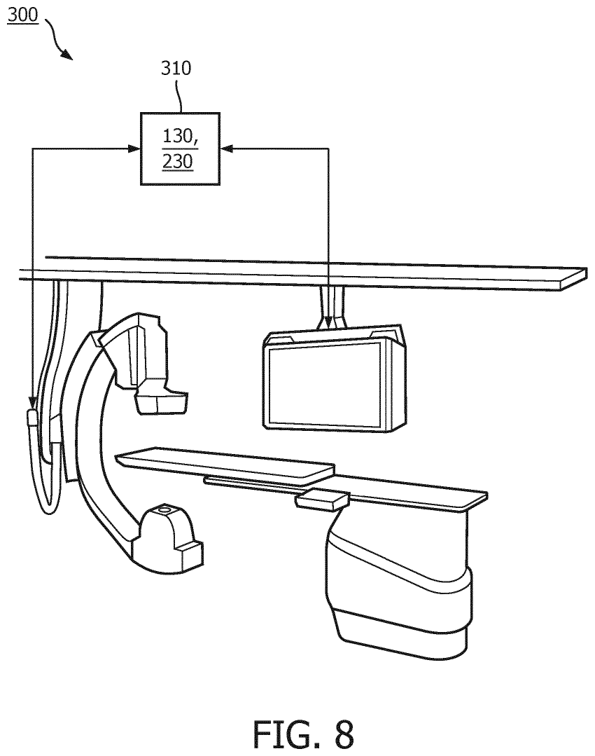

In accordance with another example, a system 300 for providing an endovascular coil specification for treating an aneurysm in a coil embolization procedure, is provided. FIG. 8 is a schematic diagram illustrating an example system 300 for providing an endovascular coil specification for treating an aneurysm in a coil embolization procedure, in accordance with some aspects of the disclosure. The system 300 include one or more processors 310 that are configured to perform one or more of the aforementioned methods. The system 300 may be used in combination with, or include, an X-ray imaging system such as that illustrated in FIG. 8. The system may also include one or more computer-readable storage media (not illustrated in FIG. 8) that include instructions for performing the method, and/or one or more displays as illustrated in FIG. 8, and/or a user interface device (not illustrated in FIG. 8) such as a keyboard, and a pointing device such as a mouse for controlling the execution of the method, and/or a patient bed, as illustrated in FIG. 8.

In accordance with another example, a computer-implemented method of training a neural network for providing an endovascular coil specification of an endovascular coil for treating an aneurysm in a coil embolization procedure, is provided. The training method includes:
receiving S210 X-ray image training data 210, the X-ray image training data 210 comprising one or more X-ray images including an aneurysm 120;
receiving S220 ground truth endovascular coil specification data 220 representing endovascular coil data of an endovascular coil used to treat the aneurysm 120 in the X-ray image training data 210;
inputting S230 the received X-ray image training data 210, into the neural network 130, 230, and adjusting S240 parameters of the neural network 130, 230 based on a loss function representing a difference between the endovascular coil data 140, 150, predicted by the neural network 130, 230, and the endovascular coil data of the endovascular coil used to treat the aneurysm 120 in the X-ray image training data 210 represented by the received ground truth endovascular coil specification data 220.

Other aspects disclosed above in relation to the training of the neural networks 130, 230, may also be included in the training method.

The above examples are to be understood as illustrative of the present disclosure and not restrictive. Further examples are also contemplated. For instance, the examples described in relation to the computer-implemented method, may also be provided by a computer program product, or by a computer-readable storage medium, or by the system 300, in a corresponding manner.

It is to be understood that a feature described in relation to any one example may be used alone, or in combination with other described features, and may also be used in combination with one or more features of another of the examples, or a combination of other examples. Furthermore, equivalents and modifications not described above may also be employed without departing from the scope of the invention, which is defined in the accompanying claims. In the claims, the word “comprising” does not exclude other elements or operations, and the indefinite article “a” or “an” does not exclude a plurality. The mere fact that certain features are recited in mutually different dependent claims does not indicate that a combination of these features cannot be used to advantage. Any reference signs in the claims should not be construed as limiting their scope.

1. A computer-implemented method of providing an endovascular coil specification of an endovascular coil for treating an aneurysm in a coil embolization procedure, the method comprising:
receiving X-ray image data comprising a plurality of X-ray images including an aneurysm, the plurality of X-ray images representing different steps of the coil embolization procedure;
extracting at least one image feature of the aneurysm from the plurality of X-ray images;
predicting, from the at least one extracted image feature of the aneurysm, endovascular coil data comprising an endovascular coil specification of an endovascular coil to be used in a next step of the coil embolization procedure for treating the aneurysm; and
outputting the endovascular coil specification.

2. The computer-implemented method according to claim 1, wherein the X-ray image data comprises at least one of one or more X-ray fluoroscopy images and one or more contrast-enhanced X-ray images.

3. The computer-implemented method according to claim 1, wherein the plurality of X-ray images at least one of i) comprises multiple different viewing angles of the aneurysm and ii) represents different timesteps during the coil embolization procedure.

4. The computer-implemented method according to claim 1, further comprising:
segmenting the X-ray image data to identify the aneurysm and extract the at least one image feature of the aneurysm to predict the endovascular coil data.

5. The computer-implemented method according to claim 1, wherein the endovascular coil data includes:
at least one of i) one or more endovascular coil parameters for treating the aneurysm and ii) one or more characteristics of an endovascular coil for treating the aneurysm;
wherein the one or more endovascular coil parameters are selected from the group consisting of: a coil length, a coil diameter, a coil stiffness, and a coil loop diameter;
and wherein the characteristics of the endovascular coil are selected from the group consisting of: a coil type and a coil material.

6. The computer-implemented method according to claim 1, wherein a neural network is trained to predict, from the at least one extracted image feature, the endovascular coil data of the endovascular coil, by:
receiving X-ray image training data comprising one or more X-ray images including an aneurysm;
receiving ground truth endovascular coil specification data representing endovascular coil data of an endovascular coil used to treat the aneurysm in the X-ray image training data; and
inputting the received X-ray image training data into the neural network, and adjusting parameters of the neural network based on a loss function representing a difference between the endovascular coil data, predicted by the neural network, and the endovascular coil data of the endovascular coil used to treat the aneurysm in the X-ray image training data represented by the received ground truth endovascular coil specification data.

7. The computer-implemented method according to claim 6, wherein the neural network is trained to predict the endovascular coil data of the endovascular coil for treating the aneurysm, from the X-ray image training data, and from volumetric image training data representing the aneurysm in the X-ray image training data; and wherein the neural network is trained to predict the endovascular coil data of the endovascular coil, by further:
receiving the volumetric image training data representing the aneurysm in the X-ray image training data;
inputting the received volumetric image training data into the neural network; and
predicting the endovascular coil data of the endovascular coil for treating the aneurysm, from the received X-ray image training data, and from the received volumetric image training data.

8. The computer-implemented method according to claim 6, wherein the neural network is further trained to predict the endovascular coil data of the endovascular coil for treating the aneurysm, from patient training data corresponding to the aneurysm in the X-ray image training data; and wherein the neural network is trained to predict the endovascular coil data of the endovascular coil for treating the aneurysm, by further:
receiving the patient training data corresponding to the aneurysm in the X-ray image training data;
inputting the received patient training data into the neural network; and
predicting the endovascular coil data of the endovascular coil for treating the aneurysm, based further on the received patient training data.

9. The computer-implemented method according to claim 6, wherein the neural network is further trained to predict the endovascular coil data of the endovascular coil for treating the aneurysm, by further;
receiving ground truth procedural outcome data representing an outcome of using the ground truth endovascular coil specification data to treat the aneurysm in the X-ray image training data;
wherein the adjusting of the parameters of the neural network comprises reducing a value of the loss function; and
wherein a negative procedural outcome is configured to increase a value of the loss function.

10. The computer-implemented method according to claim 6, wherein the neural network is further trained to predict, from the X-ray image data, procedural outcome data representing at least one of:
a fractional value representing the completeness of the coil embolization procedure;
a risk of rupture of the aneurysm;
a risk of recanalization of the aneurysm;
a recommended follow-up interval;
consequent to using the predicted endovascular coil data of the endovascular coil to treat the aneurysm in the coil embolization procedure;
and wherein the neural network is trained to predict the procedural outcome data by:
receiving ground truth procedural outcome data representing an outcome of using the ground truth endovascular coil specification data to treat the aneurysm in the X-ray image training data; and
inputting the received ground truth procedural outcome data, into the neural network;
and wherein the loss function used in the adjusting parameters of the neural network is based further on a difference between the procedural outcome data predicted by the neural network, and the received ground truth procedural outcome data.

11. The computer-implemented method according to claim 1, further comprising comparing the outputted endovascular coil data with a database of endovascular coil data for each of a plurality of endovascular coils; and
identifying one or more of the plurality of endovascular coils for treating the aneurysm in the coil embolization procedure, based on the comparing.

12. The computer-implemented method according to claim 1, further comprising computing a confidence estimate of the endovascular coil data predicted by the neural network.

13. A system for providing an endovascular coil specification for treating an aneurysm in a coil embolization procedure, the system comprising: to claim 1
a processor communicatively coupled to memory, the processor configured to:
receive X-ray image data comprising a plurality of X-ray images including an aneurysm, the plurality of X-ray images representing different steps of the coil embolization procedure;
extract at least one image feature of the aneurysm from the plurality of X-ray images;
predict, from the at least one extracted image feature of the aneurysm, endovascular coil data comprising an endovascular coil specification of an endovascular coil to be used in a next step of the coil embolization procedure for treating the aneurysm; and
output the endovascular coil specification.

14. The computer-implemented method of claim 6, wherein the neural network is trained by:
receiving X-ray image training data comprising a plurality of X-ray images including an aneurysm, the plurality of X-ray images including pre-procedural X-ray images and intra-procedural X-ray images representing the different steps during the coil embolization procedure;
receiving ground truth endovascular coil specification data representing endovascular coil data of an endovascular coil used to treat the aneurysm in the X-ray image training data; and
inputting the received X-ray image training data, into the neural network, and adjusting parameters of the neural network based on a loss function representing a difference between the endovascular coil data, predicted by the neural network, and the endovascular coil data of the endovascular coil used to treat the aneurysm in the X-ray image training data represented by the received ground truth endovascular coil specification data.

15. A non-transitory computer-readable storage medium having stored a computer program comprising instructions which when executed by a processor cause the processor to:
receive X-ray image data comprising a plurality of X-ray images including an aneurysm, the plurality of X-ray images representing different steps of the coil embolization procedure;
extract at least one image feature of the aneurysm from the plurality of X-ray images;
predict, from the at least one extracted image feature, endovascular coil data comprising an endovascular coil specification of an endovascular coil to be used in a next step of the coil embolization procedure for treating the aneurysm; and
output the endovascular coil specification.

16. The method according to claim 1, further comprising applying a machine-learning model trained to predict the endovascular coil specification of an endovascular coil to be used in a next step of the coil embolization procedure based on the at least one extracted image feature of the aneurysm, wherein the machine-learning model is trained to correlate image features of aneurysms to characteristics of different endovascular coils for treating the aneurysms in different steps of coil embolization procedures.

17. The method according to claim 1, wherein the at least one extracted image feature of the aneurysm includes at least one of aneurysm bifurcation, aneurysm size, aneurysm position, aneurysm angle relative to the blood flow, curvature of a parent vessel, and aneurysm neck diameter.

18. The system according to claim 13, wherein the processor is further configured to:
apply a machine-learning model trained to predict the endovascular coil specification of an endovascular coil to be used in a next step of the coil embolization procedure based on the at least one extracted image feature of the aneurysm, wherein the machine-learning model is trained to correlate image features of aneurysms to characteristics of different endovascular coils for treating the aneurysms in different steps of coil embolization procedures.

19. The system according to claim 13, wherein the at least one extracted image feature of the aneurysm includes at least one of aneurysm bifurcation, aneurysm size, aneurysm position, aneurysm angle relative to the blood flow, curvature of a parent vessel, and aneurysm neck diameter.

20. The non-transitory computer-readable storage medium according to claim 15, wherein the instruction, when executed by the processor, further cause the processor to:
apply a machine-learning model trained to predict the endovascular coil specification of an endovascular coil to be used in a next step of the coil embolization procedure based on the at least one extracted image feature of the aneurysm, wherein the machine-learning model is trained to correlate image features of aneurysms to characteristics of different endovascular coils for treating the aneurysms in different steps of coil embolization procedures.
